(19) United States
(12) Patent Application Publication
(10) Pub. No.: US 2008/0003183 A1
(43) Pub. Date: Jan. 3, 2008

(54) Nanoparticle radiosensitizers

(75) Inventor: Ting Guo, Davis, CA (US)

Correspondence Address: MORRISON & FOERSTER LLP, 425 MARKET STREET, SAN FRANCISCO, CA, 94105-2482, US

(73) Assignee: THE REGENTS OF THE UNIVERSITY OF CALIFORNIA, Oakland, CA (US)

(21) Appl. No.: 11/728,943

(22) Filed: Mar. 26, 2007

Related U.S. Application Data
(63) Continuation-in-part of application No. PCT/US05/34949, filed on Sep. 27, 2005.
(60) Provisional application No. 60/614,137, filed on Sep. 28, 2004.

(51) Int. Cl. A61K 49/04; A61K 33/24; A61P 31/18; A61P 35/00; A61K 39/395

(52) U.S. Cl. 424/9.42; 424/178.1; 424/617; 424/649; 977/810

(57) ABSTRACT

Herein is described Nanostructure Enhanced X-ray Therapy (NEXT), which uses nanomaterials as radiosensitizers to enhance electromagnetic radiation absorption in specific cells or tissues. The nanomaterial radiosensitizers emit Auger electrons and generate radicals in response to electromagnetic radiation, which can cause localized damage to DNA or other cellular structures such as membranes. The nanomaterial radiosensitizers contain moieties for specific targeting to molecules or structures in a cell or tissue, and can be functionalized for increased stability and solubility. The nanomaterial radiosensitizers can also be used as detection agents to help in early diagnosis of disease. Together with known techniques such as Computed Tomography or Computerized Axial Tomography (CT or CAT scan), these nanomaterial radiosensitizers could allow early diagnosis and treatment of diseases such as cancer and HIV.

### RELATED APPLICATIONS

This application is a Continuation-in-Part of International Application number PCT/US2005/034949, filed Sep. 27, 2005, which claims the benefit under 35 USC § 119(e) of U.S. Provisional application No. 60/614,137, filed Sep. 28, 2004.

### GOVERNMENT RIGHTS

This invention was made with U.S. Government support from the National Science Foundation grant number 0135132. The U.S. Government may have certain rights in this invention.

### FIELD OF THE INVENTION

Described herein are compositions, devices and methods for use as radiosensitizers, particularly the use of nanomaterials as radiosensitizers for therapy and diagnosis of diseases such as cancer and HIV.

### BACKGROUND OF THE INVENTION

The toxicity of x-rays to biological species has been the cornerstone of cancer treatment for decades (Moss et al., 2003 and Hall et al., 1973). However, x-rays alone are an ineffective modality because they lack the selectivity toward killing malignant cells while sparing healthy ones. To make x-rays more effective therapeutically, a great deal of effort has been expended in search for x-ray radiation sensitizers, which can increase the ability of x-rays to kill tumor cells while reducing their toxicity to healthy cells.

It is believed that a major source of toxicity of ionizing x-rays originates from the secondary species such as secondary electrons, including Auger electrons, and radicals generated in aqueous solutions (von Sonntag, 1987). Auger or other secondary electrons can either interact with water molecules to produce radicals that will eventually react to break the backbone of the DNA (Walicka et al., 2000, Charlton et al., 1981); or they can effectively cause single- and double-strand breaks (SSB and DSB) in DNA through direct interactions (Boudaiffa et al., 2000).

The ultimate goal is to create the so-called “Magic Bullet” that would only damage the DNA in the tumor cells while sparing the healthy ones. In doing so, the absorption of x-rays by water and other light elements in normal cells must be minimized while increasing the absorption of x-rays by the malignant cells. However, this is a challenge because water molecules still absorb x-rays at very high x-ray energies through Compton scattering (Agarwal et al., 1991). Therefore, the goal is transformed into finding efficient radiosensitizers that will selectively absorb certain x-rays at whose energy water absorbs only weakly.

Many chemicals and schemes have been employed and developed toward achieving this transformed goal. For example, in photon activated therapy (PAT) iodinated deoxyuridine (IdUrd or IUdR) analogs are used to replace thymidine in the nuclear DNA, and the DNA samples are irradiated with x-rays of energy just above the K absorption edge (˜33.2 keV) of iodine (Fairchild et al., 1984, Laster et al., 1993). Because the absorption of a hard x-ray photon by each iodine atom can lead to the release of 20-30 secondary electrons, PAT is highly effective in terms of damaging the nuclear DNA (Karnas et al., 2001, Hofer et al., 2000). In combination with computed Computed Tomography or Computerized Axial Tomography (CT or CAT scan), binary approaches such as PAT can potentially become a powerful cancer treatment method. However, the results of clinical trials of PAT have been disappointing, possibly due to rapid clearance of IUdR from the blood and the low level of incorporation of IUdR in the tumor sites (4% detected in vivo whereas 20% was used to demonstrate the effectiveness in vitro) (Kinsella et al., 1988). Another problem with this method is that the penetration depth of 34 keV x-rays is only a few mm into the body; at this energy water and bone still absorb x-rays intensely, as shown in FIG. 9.

Other chemotherapeutic agents such as platinum salts have also been found to function as radiosensitizers (Pignol et al., 2003, Kobayashi et al., 2003). However, it has been suggested that their sensitizing ability may not originate from the Auger or other secondary electrons. Similarly, radiotherapy using radionuclides has been a vigorous field for decades, and some recent developments have made it even more potent (Chen et al., 2003, Kassis et al., 2003). This method, however, lacks the double-latched safety protocol of employing radiosensitizers and external radiation. There is thus a need for new therapeutic and diagnostic tools and methods which can improve on both the specificity and efficacy of current technology.

### SUMMARY OF THE INVENTION

The present invention meets this need by providing a new type of radiosensitizer that utilizes nanomaterials targeted to cellular structures and molecules such as DNA. This method is called Nanostructure Enhanced X-ray Therapy (NEXT). In this method, nanomaterials of heavy elements such as gold are used to enhance x-ray absorption; and chemically targeted delivery of the nanomaterials to the target sites enhances specificity. In addition, x-ray beams such as those used in Computed Tomography or Computerized Axial Tomography (CT or CAT scan) and multiple collimated x-ray beams used in Gamma Knife technology can be used to further enhance the specificity of both detection and treatment of disease.

There are several distinct advantages to this methodology. First, heavy elements such as gold absorb more high energy x-rays, thus increasing the linear energy transfer (LET) from x-rays to the target. Second, although nanostructures such as a nanoparticle absorbs the same amount of x-rays as the same number of individual atoms, the former helps to localize the toxicity of the x-rays to the nanoparticle site by emitting an intense burst of Auger electrons and other secondary electrons from a single nanoparticle. These low energy electrons are highly localized at the targeted sites, thus producing high concentrations at these sites. This means the effective LET is even higher for gold nanoparticles than gold atoms spread over a much larger volume in solution. Third, if gold nanoparticles are used, the x-ray energy to be used would be above 81 keV (the K absorption edge of gold), leading to less absorption by light elements in the body and thus deeper penetration. Fourth, smaller nanoparticles, patterned aggregates of small nanoparticles, or other materials such as gold nanorods or nanobeads with a higher percentage of surface atoms offer the Auger or secondary electrons an easier escape with minimal residual positive charges on each individual nanoparticles. Fifth, gold is one of the most benign elements to the body, although gadolinium, gold, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu) iodine, tungsten, rhenium, osmium, iridium, platinum, and bismuth may also be utilized. Lastly, nanoparticles have been modified chemically and used in many cases to connect to DNA segments, so it is possible to extend this method to tackle specific sites (Shenhar et al., 2003, Alivisatos et al, 1996, McDevitt et al., 2000, Zanchet et al., 2002, Sandstrom et al., 2003).

Described herein is a method of inducing damage to a molecule, including the steps of: delivering a nanomaterial or nanostructure including at least one targeting moiety capable of binding to the molecule to a location that is 5-10 nm or less in distance from the molecule and exposing the nanomaterial to electromagnetic radiation under conditions wherein the nanomaterial releases electrons that directly or indirectly induce damage to the molecule. These electrons and especially the radicals generated by them will cause damage to DNA, cellular membranes, or other targets.

It is important to target these nanostructures to the biological sites, preferably within 5-10 nm of the targets. Conjugation of these nanostructures to the targets is important when only very small amounts of nanomaterials are employed. When conjugation is used, the amount of nanostructures required for enhanced damage is typically between 0.1% and 0.0001% of the cell mass. On the other hand, much greater amounts of nanostructures are needed when there is not direct chemical conjugation between them and the biological targets. Typically, the lower limit of the weight percentage of the nanostructures is 1%, which makes it difficult to implement clinically. In most cases, such implementation can even lead to anti-enhancement and other side effects because of the presence of a large amount of nanostructures and chemical ligands that can scavenge the electrons and radicals generated in water. These electrons and radicals constitute the damage to the targets without the introduction of the nanostructures.

The nanomaterials can be selected from nanoparticles, nanorods, nanoshells, nanowires, complexes consisting of nanoparticle-nanorod and nanoparticle-nanowire combinations, patterned nanoparticles chemically linked by ligands, or silicon-based nanowires (SiNW). The shape of the nanomaterials can be spherical, cylindrical, donut-like, wire-like, needle-like, star-like, beads-on-a-string-like, or balloons-in-one-hand-like. The nanomaterials can be made of a heavy metal such as gadolinium, gold, lanthanum (La), cerium (Ce), praseodymium (Pr), neodymium (Nd), promethium (Pm), samarium (Sm), europium (Eu), gadolinium(Gd), terbium (Tb), dysprosium (Dy), holmium (Ho), erbium (Er), thulium (Tm), ytterbium (Yb), lutetium (Lu), iodine, tungsten, rhenium, osmium, iridium, platinum, and bismuth. The nanoparticles made of these heavy elements may also contain a small percentage of lighter elements such as iron or silicon on the surface of the nanoparticles for the purpose of forming stronger bonds between the surface atoms on the nanoparticles and the moieties and ligands chosen for targeting and functionalization. The nanomaterials can be further surrounded by a layer of UV (ultraviolet) to IR (infrared) chromophores or fluorophores, such as tryptophan and ethidium.

The complex nanostructures such as strings of nanoparticles at one location can be used to increase double strand breaks (DSBs), which are much more difficult to repair than single strand breaks. Again, it is important to chemically conjugate a small number of nanoparticles to the target to achieve enhanced damage.

The nanomaterials can be functionalized to increase solubility, stability and/or biocompatibility. Some examples of ligands which can be used to functionalize the nanomaterials include: triethylammonium (TMA), ethoxy ligands, (poly) ethoxy thiol, amine thiol, ammonium thiol, mercaptoundecanoic acids, phosphanes/PPh₃, single or double strand DNA segments, Dextran, thiolated oligonucleotides, carboxylic thiols, PVA, polyglycols, and ester thiols. The ligands can be crosslinked.

If nanoparticles are used, the shape of the nanoparticles can be spheres, cylindrical rods and disks. The nanoparticles can range in size from 1 to 1000 nm, or 1 to 15 nm.

The molecule in or on a cell or tissue can be, for example, DNA, proteins, capillary vessels, or viruses. The cell to which the nanomaterial radiosensitizer is delivered can be, for example, a cancer cell, a bacterial cell or a cell infected with a virus.

The binding or bonding described above, between the moiety and the molecule in or on a cell or tissue, can be by covalent bonds, electrostatic interactions, hydrogen bonds, van der Waals interactions, dispersion forces, hydrophilic/hydrophobic interactions, or magnetic forces.

The moiety which is used to target the nanomaterial radiosensitizer of the invention can be DNA capable of preferentially associating with the target DNA, antibodies, cell penetrating peptides, translocation proteins, ethidium ligands, thiol and phosphane ligands, signal peptide sequences, super-antibodies, chemotherapeutic agents, low density lipoproteins, capillary-binding molecules, or polymers.

The electromagnetic radiation to which the nanomaterial radiosensitizers are exposed can be X-ray radiation, optionally of energy greater than about 50.3 or 80.7 keV for use with gold nanomaterials. Catheters emitting low energy x-rays between 11.92. keV and 50 keV may also be employed through surgery.

A preferred embodiment is where the nanomaterial is a gold nanoparticle, the electromagnetic radiation is X-ray radiation, and the molecule is DNA.

Also described is a method of inducing damage to a bacterium, bacterial nucleic acid, virus or viral nucleic acid sequence in a cell or tissue including the steps of delivering nanoparticles to the cell or tissue; and exposing the nanoparticles to electromagnetic radiation under conditions where the nanoparticles release high energy photoelectrons and secondary electrons such as Auger electrons. The released Auger or other secondary electrons induce local damage to the bacterium, bacterial nucleic acid, virus or viral nucleic acid sequence. The virus or viral nucleic acid can be inside a virus or viral particle, outside the virus or viral particle and integrated into the genome of the cell, or outside the virus or viral particle and not integrated into the genome of the cell. The nanomaterials further include at least one moiety which specifically binds or bonds to the virus or viral nucleic acid sequence.

Also described herein is a method of detecting a cancer cell, a bacterium, a bacterial nucleic acid, a virus, or a viral nucleic acid sequence including delivering nanomaterials to a cell or tissue and exposing the nanomaterials to electromagnetic radiation under conditions where the nanoparticles emit radiation, and then detecting the emitted radiation with a detector to detect the cancer, bacterium, bacterial nucleic acid, virus, or viral nucleic acid sequence. The nanomaterials also include at least one moiety which targets the nanomaterials to the cancer cell, bacterium, bacterial nucleic acid, virus, or viral nucleic acid sequence. An external x-ray detector, for example those used in detection of radiation from radionuclides, can be used in combination with a collimated x-ray beam in the scanning mode. As an example, the nanomaterials can emit characteristic K line x-rays after absorption of external x-rays. The characteristic x-rays can be detected by the detector. In this case, the energy of the external excitation x-rays must also be higher than the absorption edge of the elements in the nanoparticles.

Also described herein is method of damaging a cell or tissue including the steps of delivering nanomaterials coated in UV chromophores or fluorophores, such as tryptophan, to a cell or tissue; and exposing the nanomaterials to electromagnetic radiation under conditions wherein the nanomaterials release Auger or other secondary electrons, which interact with the chromophores or fluorophores to produce UV light upon exposure to electromagnetic radiation, which damages said cells or tissues.

Also described herein is a cancer cell, bacterial cell or virus detection or inactivation agent including a nanomaterial capable of emitting Auger or other secondary electrons when exposed to electromagnetic radiation. The nanomaterial also includes at least one moiety which targets the nanomaterial to the cancer cell or virus. Also described herein is a pharmaceutical composition including the cancer cell or virus detection or inactivation agent in association with one or more pharmaceutically acceptable carriers.

### BRIEF DESCRIPTION OF THE DRAWINGS

FIG. 1A shows Transmission Electron Microscopy (TEM) analysis of gold nanoparticles. The size distribution is shown in the left panel. The scale bar is 10 nm. FIG. 1B shows TEM images of gold nanoparticles made with slow injection of 1× concentration of alkanethiol ligands (left panel) versus fast injection (10 sec) of abundant (2× concentration) alkanethiol ligands (right panel). FIG. 1C shows a TEM image of nanoparticles after ligand exchange reactions in DCM. Several large nanoparticles are still visible, although the result shows a large number of uniformly sized small nanoparticles.

FIG. 2 shows Agarose gels (0.8%) after electrophoresis of TMAₙAuNP and scDNA. The distances between the two columns of wells were different for lanes 1-5 and lane 6. Lanes are labelled as mA (left column) or mB, where m is the lane number 1 through 6. The polarity of the gels is shown with the + and − signs. 1.65 μg 51-hour TMAₙAuNP in 10 μL Milli-Q (MQ) water was added into wells 2A, 3A, 5A, and 6A. 1 μg scDNA in 8 μL MQ water was added into wells 1A, 4B, 5B, and 6B. A 1:1000 ratio mixture of scDNA (1 μg scDNA in 8 μL MQ water) to TMAₙAuNP (16.5 μg 51-hour AuNP in 10 μL MQ water) was added into well 2A. Wells 1B, 2B, 3B, and 4A were left empty.

FIG. 3A shows results from the E-gels from the radiation testing. The duration of the irradiation is shown under the bands in the gel. The AuNP-to-scDNA ratio was ˜100:1. Magnified bands of lane 3 and 9 are shown. The ladders are in lane 6. FIG. 3B shows the lineout plots of another gel. The samples were prepared similarly to those in FIG. 1A. Vertical lines are drawn for visual alignment of the bands.

FIG. 4 shows statistics of the gels from FIG. 2 on the radiation tests. FIG. 4A shows the percentage of the relaxed form. scDNA (empty symbols) and AuNP-scDNA (corresponding solid symbols) are shown. FIG. 4B shows the relative enhancement ratios as a function of radiation dosage for three sets of samples.

FIG. 5 shows an ethidium ligand with a short alkane chain (top panel). The lower panel shows the enhancement of fluorescence when quenched ethidium connected to gold nanoparticles becomes fluorescent again.

FIG. 6 shows examples of three possible types of ligand-nanoparticle complexes (A, B, and C) to make more stable ligand-covered nanoparticles for NEXT applications.

FIG. 7 shows the results of coating silicon-based nanowires with gold nanoparticles. The left panel shows the gold nanoparticles as made. The right panel shows the nanoparticle-nanowire complexes. The gold nanoparticles are covered with citrate groups, and the nanowires are covered with amino-propyl-triethoxy-silane (ATPS). The interaction is electrostatic.

FIG. 8 shows a schematic diagram of detection of Auger electrons using nanoparticle-nanowire composite materials.

FIG. 9 shows x-ray absorption by water, gold (top panel) and bone (lower panel) for x-rays whose energy is between 1 keV to 100 MeV.

FIG. 10 shows a schematic drawing of three types of nanomaterials that can be used as radiosensitizers. They include a nanorod, a string of nanoparticles, and individual nanoparticles.

FIG. 11 shows how NEXT agents can be used to absorb incoming x-rays in a scanning x-ray beam and emit characteristic x-ray fluorescence for CT-like detection and image reconstruction. After each scan, the assembly of the source and detector is moved to the next position for another scan. The signals are stored and processed by computers for 3-D image reconstruction.

FIG. 12 shows Transmission electron microscope image of scDNA with gold nanoparticles conjugated to them. The scDNA were stained to increase contrast (A). Also shown are the results of DNA damage as a function of buffer concentration, which directly controlled the diffusion distance of OH radicals (B).

FIG. 13 shows Scanning electron microscopy (SEM) images of gold nanotubule matrix for scDNA radiation experiments (A) and enhancement of radiation damage to scDNA in the gold nanotubule matrix as a function of buffer concentration (B).

### DETAILED DESCRIPTION OF THE INVENTION

Described are compositions and methods that may be used to treat or detect various diseases and conditions. The compositions described include a nanomaterial, coupled to one or more moieties which provide targeting to one or more cells, molecules or structures in a cell or tissue. The nanomaterials of the invention are also referred to herein as nanomaterial radiosensitizers. The use of the materials of the invention is also referred to as Nanostructure Enhanced X-ray Therapy (NEXT). In the methods, one or more components of a composition are associated with one or more molecules or structures, and upon exposure to electromagnetic radiation the composition components release photoelectrons, Auger electrons, and secondary electrons, which generate radicals and other species such as ultraviolet (UV) to infrared (IR) photons. The Auger or secondary electrons and radicals generated by these electrons then damage the targeted cells, molecules or structures or cells. Under some circumstances the Auger or other secondary electrons and radicals can damage molecules and structures within approximately 10 nm of the nanomaterial releasing the Auger or other secondary electrons. Without chemical conjugation, damage to the target is mainly achieved with photoelectrons and radicals generated from these electrons. These nanomaterials, once targeted to a molecule or structure of interest, can also be detected after absorption of x-rays. This invention thus provides a powerful tool for early detection and effective treatment of diseases such as cancer, bacterial infections, and viruses such as HCV and HIV.

Nanomaterials

Generally, any composition may be used that releases Auger or other secondary electrons upon exposure to electromagnetic radiation of appropriate energy.

Compositions

Compositions of the nanomaterial radiosensitizers can vary, as long as the nanomaterial radiosensitizer is capable of emitting Auger or other secondary electrons to damage cells, molecules or structures in or on a cell or tissue when exposed to electromagnetic radiation of appropriate energy.

The nanomaterial radiosensitizers may be made of a pure material. Mixtures of other than pure precursors can be used to make composite nanomaterial radiosensitizers.

Heavy metal nanomaterials may be used. Gadolinium nanomaterials may be used. Nanomaterials made of Pt, Bi and their alloys can be used. Nanomaterials made of alloys of other elements such as Si, Fe, I and Br with Au, gadolinium, gold, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), iodine, tungsten, rhenium, osmium, iridium, platinum, and bismuth and their alloys may also be used. Gold nanomaterials are preferred, as gold has low toxicity. Additional elements other than heavy elements may be used to increase ligand stability on the surface of the nanomaterials. For example, a small percentage of Pd may be added to the nanomaterials to increase the stability of thiol or other surface ligands on gold nanoparticles (Nutt et al., 2005).

Shape and Size

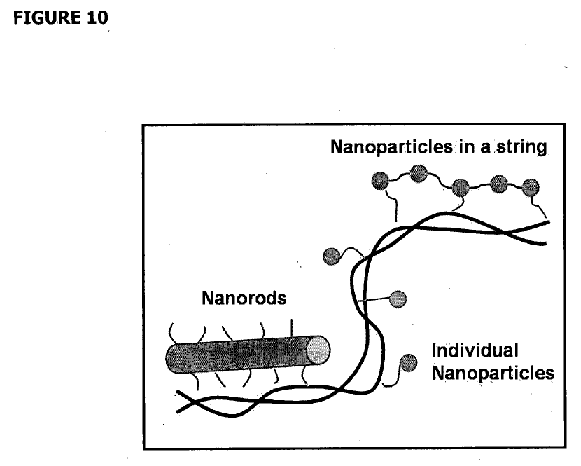

Generally, the nanomaterial radiosensitizers may be of any geometry and dimension that allows release of Auger or other secondary electrons upon exposure to electromagnetic radiation. The shape of the nanomaterial radiosensitizers can be for example spherical, cylindrical, circular or donut-like, wire-like, needle-like, star-like, “beads-on-a-string-like”, or “balloons-in-one-hand-like” (Shenhar and Rotello, 2003). The object is to concentrate as much material in the smallest volume to enhance the absoption of electromagnetic radiation while maximizing the percentage of the surface atoms to facilitate the release of Auger or other secondary electrons. A schematic drawing of three types of nanomaterials is given in FIG. 10. In the drawing, a nanorod, a string of nanoparticles, and several individual nanoparticles are shown. The density of Auger or other secondary electrons, which is proportional to the amount of surface area, is higher for the nanorod and the string of nanoparticles as compared to the individual nanoparticles.

Nanomaterials that can be used in the present invention are therefore not limited to solid spherical nanoparticles. Other nanomaterials such as nanorods, nanoshells, short nanowires, complexes consisting of nanoparticle-nanorod and nanoparticle-nanowire combinations, patterned nanoparticles connected by ligands such as dendrimers and nanowires such as silicon-based nanowires (SiNW) are possible choices that can be used to enhance x-ray absorption and increase localized x-ray damage to biological samples such as tumor cells (Nikoobakht and El-Sayed, 2003, Shenhar and Rotello, 2003, Zanchet et al., 2002, Sandstrom et al., 2003).

The size of the nanomaterial radiosensitizers will be determined by their accessibility to the cell and cellular nuclei. Generally it ranges from 1 nm to 1000 nm. If the nanomaterials are approximately spherical, a composition of nanomaterials with average diameter of between about 1 nm and about 15 nm may be used. Nanomaterials with a linear dimension of a few nanometers or less may be used.

As an example, spherical or near spherical pure gold nanoparticles ranging from 2 to 20 nanometers in diameter were used in the example. These gold nanoparticles consisted of a solid gold core. The surface of the nanoparticles was covered with a mixture of alkanethiol and trimethylammonium thiol ligands. The alkanethiol functions as a protective layer for the nanoparticles, and the trimethylammonium thiol ligands make the nanoparticles more soluble in water and target DNA through electrostatic interactions.

Nanomaterial Production

There are various methods by which the nanomaterial radiosensitizers of the invention can be made. One method is a two phase reaction in which an aqueous solution of gold compounds such as hydrogen tetrachloroaurate is mixed with a solution of tetraoctylammonium bromide in toluene. After mixing, dodecanethiol protection ligands are injected into the two-phase solution. Sodium boronhydride is then added to reduce gold ions for gold nanoparticles (Brust et al, 1994). Different ratios of aurate and thiols can be used (Wang et al, 2002). Gold nanoshells have been made by Halas et al, which can also be used as NEXT agents (Loo et al, 2005). Nanorods made from gold nanoparticles seeds can also be used when they are properly ligated. (Nikoobakht et al., 2003) Small gold or palladium nanocrystals have been made from gold or palladium compounds. (Schmid et al., 1992) Inverse micelle techniques can also be used to make nanoparticles of desired sizes (Wilcoxon et al., 1999).

Composite nanomaterial radiosensitizers can be made using techniques similar to those used to make pure gold, platinum, gadolinium or bismuth nanomaterials. The methods described above with two or more precursors can be used to make composite nanoparticles. Other methods starting from one kind of nanoparticle can also be used. (Seino et al., 2004)

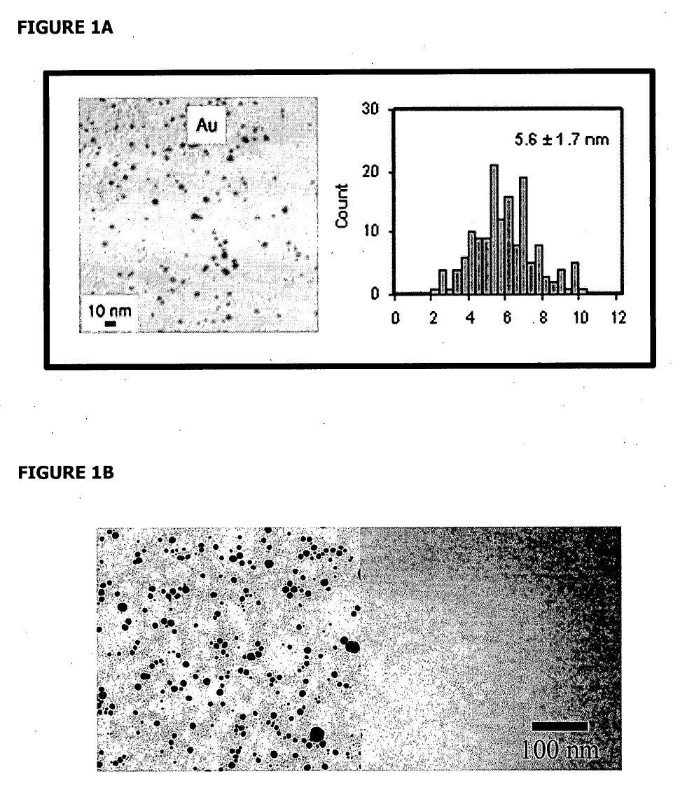

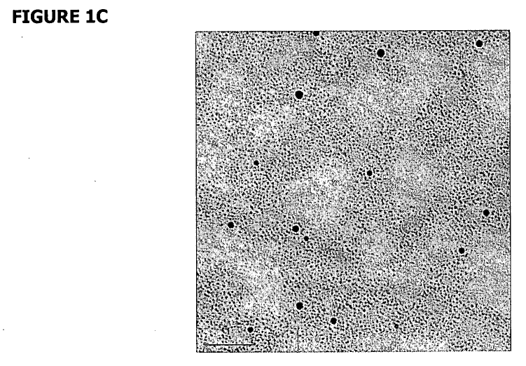

By changing the conditions such as the ratio of gold precursors and thiol ligands, it is possible to make more uniformly sized gold nanoparticles, similar to the results shown in Cliffel et al., 2000. The results of such an experiment are shown in FIG. 1B. Another method is to use different solvents to perform ligand exchange reactions. Dichloromethane (DCM), for example, can be used (Brown and Huthison, 1999). The gold nanoparticles so produced after this ligand exchange reaction, shown in FIG. 1C, are notably better than that made in water/toluene binary solvent.

Moieties for Targeting

The nanomaterials of the present invention specifically associate with molecules, structures, cells, bacteria, viruses or viral nucleic acids, etc. in or on cells or tissues. Such association is possible by including in the nanomaterial one or more moieties capable of associating the nanomaterial with the targeted molecule or structure. Such moieties include but are not limited to: DNA, antibodies, cell penetrating peptides, translocation proteins, ethidium ligands, thiol ligands, phosphane ligands, signal peptide sequences, super-antibodies, chemotherapeutic agents, low density lipoproteins, capillary-binding molecules, and polymers.

After synthesis of nanoparticles, nanorods, or other shaped nanomaterials, desired moieties are placed on the surface of these nanomaterials through ligand exchange reactions. Direct addition of the desired moieties can also be carried out during synthesis. In a preferred embodiment, the moieties are stable and have high affinities to bind to target molecules, structures, cells, viruses and viral nucleic acids, etc.

A moiety is chosen depending on the molecules, structures, cells, bacteria, bacterial nucleic acids, viruses or viral nucleic acids, etc. to be targeted. The moiety may cause association of the nanomaterial through binding or bonding to the molecule or structure to be targeted. Such binding or bonding can be through a variety of mechanisms, including but not limited to via covalent bonds, via electrostatic interactions, via hydrogen bonds, via van der Waals interactions, via dispersion forces, via hydrophilic/hydrophobic interactions, or via magnetic forces.

Currently available techniques involving radionuclides for radiation therapy and autoradiography and can be used to transport the radiosensitizers to the tumor sites or other desired places in the body. For example, ⁹⁰Y and ¹⁵³Sm connected to MULTIBONE (which is non-radioactive) radiopharmarceuticals have been used to treat bone metastases as a result of various cancers. In other examples, methionine-labeled radioactive samples or receptor (e.g., dopamine D1 and D2) ligands can be used in autoradiography. (Aldrich et al., 1992)

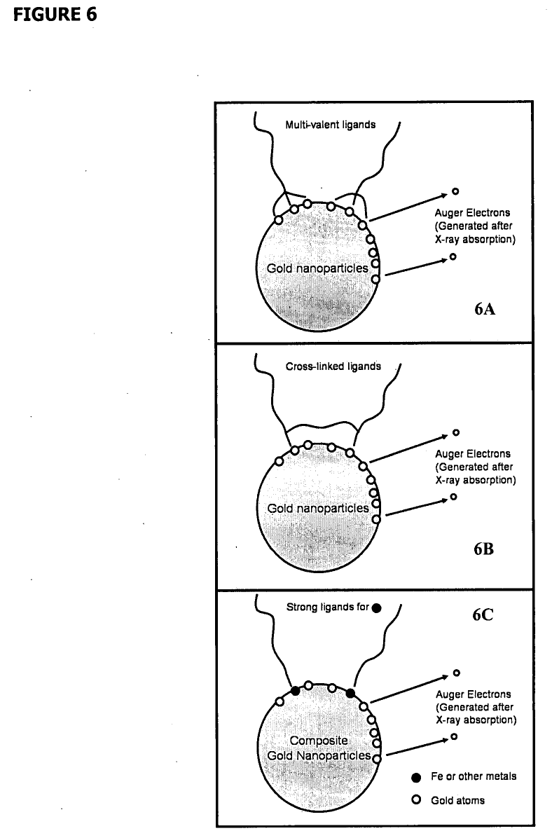

Because mono-valent moieties may make nanoparticles more reactive with each other and other species, it is possible to use multi-valent moieties (see FIG. 6A) and partially cross-linked moieties (see FIG. 6B). These multi-valent moieties provide increased stability to the nanomaterials. It is important to control the density of the moieties on the nanomaterials, because if the density is too high, it may reduce the efficacy of the Auger or other secondary electrons and radicals. In another format, composite nanoparticles-moiety complexes (see FIG. 6C) can be utilized. This format will find particular use when antibodies, proteins, or peptides are linked to the nanomaterials. Efficacy is optimized if these moieties remain attached to the nanomaterials so that an effective amount of the nanomaterials will be accumulated in the target region. If these moieties are detached from the nanomaterials, the effectiveness of NEXT can be reduced.

Provided below are examples of moieties which can be used to target the nanoparticle radiosensitizers of the present invention.

One example of using moieties to target nanomaterial radiosensitizers is to employ cell-penetrating peptides (CPP). For example, penetratin (PEN) and transportan and their analogs, and trans-acting activator of transcription (TAT) facilitate transport of large proteins into the cell (Pooga et al., 2001, Lindgren et al., 20000, Ziegler et al., 2005) CPP is powerful tool to introduce drugs into the cell (Tseng et al., 2002). CPP are useful as moieties in the present invention.

Another example is the use of protein translocation domains (PTD) linked to nanoparticle radiosensitizers (Franc et al., 2003). PTDs are peptides that breach the lipid bilayer of a cell. PTDs are useful to transport radiosensitizing nanomaterials into or onto a cell.

One can also use antibody-conjugated nanomaterial radiosensitizers to target specific antigens, including tumor sites. For example, streptavidin conjugated AuNP can be used to attach to biotin or mortalin antibody-stained cells (Kaul et al., 2003). McDevitt el al have used antibody J591 attached to ²¹³Bi for treating prostate cancer (McDevitt et al., 2000). Another antibody, anti-CD19, has been used to target lymphoma (Ghetie et al., 1994). Another antibody linked with anti-IgG-10 nm gold nanoparticles targets CD33 in stained COS7 cells (Cognet et al., 2003). These and many other antibodies can be used to target the nanomaterial radiosensitizers of the invention to specific antigens. New antibodies can be created to specific targets, which is well known in the art.

One may also use natural products (small robust antibiotics such as vancomycin, Tyrocidine A, or Paxilline) as moieties to cover the nanomaterial radiosensitizers and then deliver them to target sites, such as sites of bacterial infection or tumor sites (Bramlett et al., 2003).

Signal peptide sequences can also be attached to nanomaterial radiosensitizers to facilitate transport into the cell (Rojas et al., 1998). Signal peptide sequences are short stretches of amino acids usually found at the beginning of proteins that are typically rich in hydrophobic amino acids which helps transport through the membrane.

Super-antibodies with a short protein segment called a membrane-translocating sequence (MTS), which are normally found in signaling proteins, can penetrate the cell membrane. When bound to nanomaterial radiosensitizers, they can be used to facilitate transport of the nanomaterial radiosensitizers into the cell. The super-antibodies are generally less toxic than regular small molecule antibodies (Zhao et al., 2004). These super-antibodies may be used to target the nanomaterial radiosensitizers to bacteria and viruses, including HCV and HIV, inside infected cells.

One can also use dendritic polymer hosts to selectively bind to tumor vasculatures. Nanomaterial radiosensitizers as well as targeting moieties such as folate can be connected to these dendritic polymers (Kukowska-Latallo et al., 2005). Folic acid can be used to target solid tumors (Sovico et al., 2005; Stella et al., 2000). It is possible to decorate the dendritic particles with the nanomaterial radiosensitizers and other species to facilitate transport into the cell. Afterwards, radiation will be administered to eradicate the cell based on the mechanisms proposed herein.

Capillary-binding molecules can be attached to the nanomaterial radiosensitizers to make them target to proteins such as αVβ3, which are abundant near tumor sites (Winter et al., 2004).

Low density lipoproteins (LDL) can also facilitate transport of nanomaterial sensitizers to the surface of the tumor cells, which are subsequently taken up by the cell through endocytosis (Clark et al., 2005).

DNA capable of preferentially associating with the targeted DNA can be used as a targeting moiety when the nanomaterials are targeted to DNA (Zanchet et al., 2002). This preferential association can be facilitated through sequence-specific binding. The sequence of the DNA used as a moiety for targeting can be designed to specifically bind the target DNA sequence. One of skill in the art would readily design the moiety DNA with knowledge of the target DNA sequence.

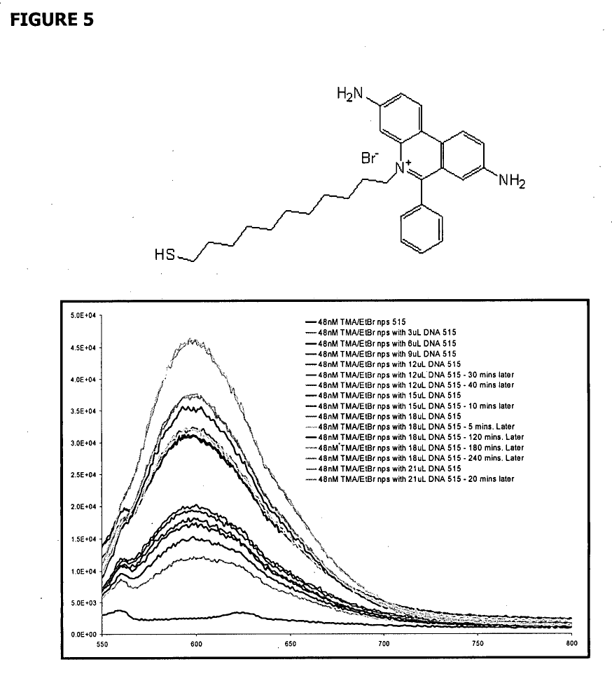

Another option is to use ethidium ligands to bind nanoparticles to DNA. Ethidium can be incorporated into gold nanoparticles (Wang et al., 2002). Ethidium, a normally fluorescent molecule that intercalates in DNA, is quenched when connected to gold nanoparticles through a short alkane chain. We have verified that these nanoparticles-ethidium complexes can intercalate into DNA, because the resulting structure becomes fluorescent again when ethidium is intercalated into DNA. These results are shown in FIG. 5. Note that almost no fluorescence was detected when ethidium is connected to gold nanoparticles. Other moieties can be used in conjunction with ethidium to first take the radiosensitizers into the cell. Once ethidium-coated radiosensitizers are in the cell, their affinity towards DNA will be enhanced through the ethidium-DNA interaction.

In another format, more than one moiety type can be incorporated into the nanomaterials. Once the nanoparticle radiosensitizers have been modified to include the moiety or moieties required, they can be delivered to a subject, as described below.

Surface Functionalization

The nanomaterials described above may further include a ligand to provide surface functionalization. Such surface functionalization may have a variety of purposes, including but not limited to increasing the water solubility of the nanomaterials, increasing the biocompatibility of the nanomaterials, increasing the solubility in fat to pass the blood brain barrier, and/or increasing their stability in an acidic environment in the body. As with the moieties discussed above, it is also important to control the density of the ligands on the nanomaterials, because if the density is too high, it may reduce the efficacy of the Auger or other secondary electrons and radicals. In some formats the ligand and the moiety may be one and the same.

There are several ligands which can be used to increase the water solubility and stability of the nanomaterials over increased periods of time. Some non-limiting examples of ligands which can be used to functionalize the surface of nanomaterial radiosensitizers to increase solubility include: (poly) ethoxy thiol, amine thiol, ammonium thiol, mercaptoundecanoic acids, phosphanes/PPh3, single or double strand DNA segments, Dextran (Neuwelt et al., 2004), thiolated oligonucleotides, carboxylic thiols, PVA, polyglycols, and Ester thiols. Ethoxy-based ligands (OEt or ethylene oxide)n (n≧2) can be used. (Foos et al., 2002) These ligands are neutral at pH 7.0. Dextran is also useful to allow the nanoparticle radiosensitizers to cross the blood-brain barrier. TMA (trimethylammonium) can be used, as is described in the Example provided.

Cross-Linking of Ligands

To make coating ligands more stable, it is possible to cross-link the coating ligands on the surface of the nanomaterial radiosensitizers (Schroedter and Weller, 2002). Nie et al have used a similar method to make stable nanoparticles in water (Smith and Nie, 2004). Amines have been used to cross link thiols to make them more stable on gold nanoparticles (Pellegrino et al., 2004).

Nanomaterial Radiosensitizer Delivery

Once the nanomaterial radiosensitizers have been modified to contain the appropriate moieties for targeting and/or functionalized on the surface, the nanomaterial radiosensitizers can be packaged in many different ways for delivery. Compositions of the present invention generally comprise an effective amount of the nanomaterial radiosensitizers of the invention in a pharmaceutically acceptable medium. The use of such media is well known in the art. For example, it can be in solid form, or dissolved in organic or inorganic solvents to form either a solution or a stable suspension.

There are many methods by which the nanomaterial radiosensitizers of the invention can be delivered. Methods of delivery include but are not limited to: parenteral, oral (enteral), intravenous, subcutaneous, intramuscular, intra-arterial, intrathecal, and intraperitoneal. Other administration methods such as topical application, sublingual, rectal, or pulmonary are all possible.

Once the nanomaterial radiosensitizers have been delivered, they will preferentially associate with the target cellular structures or cellular molecules, as described below.

Target Molecules or Structures in a Cell or Tissue

Molecules or structures on or in a cell or tissue with which the nanomaterials may be associated include but are not limited to DNA and cell membrane surface proteins, capillary vessels, bacteria, viruses, and viral nucleic acids. Viral nucleic acid sequences include DNA or RNA of the virus inside the virus or viral particle, outside the virus or viral particle and not integrated into the DNA of the cell, and outside the virus or viral particle and integrated into the DNA of the cell. Cellular structures and cellular molecules of the subject's cells as well as foreign cells such as bacterial or parasitic cells can be targeted. Viral proteins and structures can be targeted. The moieties for targeting described above can be chosen in order to target specific cellular structures or cellular molecules.

Cellular structures and cellular molecules which are specific to a certain cell type are preferred. Some examples include cellular structures and molecules which are expressed in or on cancer cells, or cells infected with a virus. The specific targeting of these cell types increases the selectivity of the methods of the present invention. Only those cells that are associated with the nanoparticle radiosensitizers will be affected by the electromagnetic radiation administered.

Cellular structures which can be targeted include but are not limited to the plasma membrane and the membranes of intracellular organelles, as well as intracellular and cell surface proteins and structures. For example, it has been shown that radiosensitizers targeting CD45 on the cell surface of leukemia cells may lead to cellular death under radiation (Matthews et al., 1997). It is possible that the nanomaterial radiosensitizers will be endocytosed if they are captured at the surface of the cell. Therefore, it is preferred to transport these radiosensitizers into the cell, and targeting the nuclear DNA is most preferred. Many of the ligands described herein can also penetrate the endoplasmic reticulum (ER) so that the nanomaterial radiosensitizers can be transported to the nucleus (Walter and Blobel 1981). The overall size of these nanomaterial radiosensitizers is small enough to allow them to freely enter the nucleus through the nuclear pores once they are in the vicinity of the nucleus.

Uses of the Nanomaterial Radiosensitizers

The compositions and methods described herein can be used to treat a variety of diseases and conditions including but not limited to cancer, HCV, and HIV, and detect target cells of interest, for example cancer or virus infected cells.

Treatment of Diseases and Conditions.

There are many diseases which can be treated by use of the nanomaterial radiosensitizers of the present invention. Any disease where specific cell types can be targeted can be treated. Cancer, bacterial infection and viral infection are three non-limiting examples of diseases which can be treated. The target cells can be in any part of the body. The nanomaterial radiosensitizers can generally cross the blood-brain boundary to enter the brain. Therefore, they can be used to detect and treat brain tumors and even infections.

The nanomaterials of the present invention can kill target cells by the release of Auger or other secondary electrons, induced by exposure to electromagnetic radiation. The released Auger or other secondary electrons damage the DNA of the targeted cancer cells. Auger or other secondary electrons can either interact with water molecules to produce radicals, or interact with DNA directly to cause single and double strand breaks. The radicals can also react with and break the backbone of DNA or cleave base pairs from DNA. These processes eventually lead to the death of the targeted cells.

It is also possible to put a layer of UV to IR chromophores or fluorophores around the nanomaterials. Upon absorption of x-rays, Auger or other secondary electrons will be released, which interact with this layer to produce light in the UV to IR spectral region. For example, tryptophan can be put on the surface. The light generated can be used to detect the location of nanoparticles using optical microscopy. The light may also damage nearby cellular structures and molecules, including but not limited to DNA and membranes. Secondary electrons, which can cause further damage, may also be produced when UV light is absorbed by molecules and structures near the nanomaterials.

In one method that may be used for the treatment of cancer, the nanomaterials are preferentially associated with a molecule or structure of cancerous cells and the released Auger or other secondary electrons damage the targeted cancer cells.

In one method that may be used for the treatment of cancer, the nanomaterials are preferentially associated with the DNA of cancerous cells and the released Auger or other secondary electrons damage the DNA of the targeted cancer cells.

In one method that may be used for the treatment of HIV, the nanomaterials are preferentially associated with virus or viral nucleic acid sequence and the released Auger or other secondary electrons damage the virus or viral nucleic acid sequence.

Detection of Target Cells

The nanoparticle radiosensitizers of the present invention can also be used as detection agents to selectively mark certain cells or cell types. The moieties for targeting described above can be used to target the nanomaterial radiosensitizers to cells or tissues of interest. Low doses of x-rays such as those in Computed Tomography or Computerized Axial Tomography (CT or CAT scan, which is already a powerful tool to detect brain, lung, liver and other cancers) can then be administered, which are absorbed by the nanoparticle radiosensitizers. Water and other heavy objects are commonly used in CAT scan to enhance contrast.

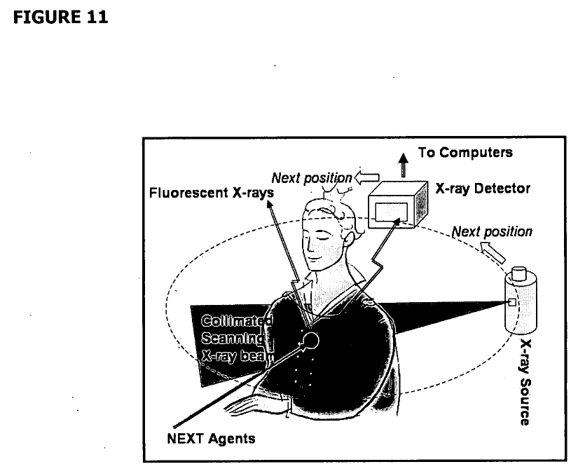

Because the release of Auger or other secondary electrons is complemented by x-ray fluorescence, x-ray emission whose wavelength is element specific also occurs after initial absorption of high energy x-rays. For example, Au emits at 68.8 keV (also called characteristic radiation) after it absorbs an x-ray photon of energy greater than 80.7 keV. When suitable targeting agents/moieties are attached to the nanomaterials, and the resulting complexes are attached to the target, they may be used as sensors that are activated only after initial x-ray radiation of low dosages. An sensitive external x-ray detector such as cesium iodide or cadmium telluride flat panel detectors or cadmium zinc telluride (CZT) solid state detectors can be used to probe the characteristic radiation. These detectors can detect x-rays from a few keV to a few MeV. In addition, wire-based x-ray detectors such as proportional counters can also be used. Using a collimated x-ray beam in the scanning mode in combination with the external detector and the targeted nanomaterial sensitizers, it is possible to increase the detection limit of diseases such as cancer and HIV at their early stages. The invention described here can be easily implemented in CT to image cancer and other diseases, except a more monochromatic x-ray source and an energy selective x-ray detector will be used instead of a generic rotating x-ray source and non-energy selective detector. Furthermore, the detector should be located away from the x-ray beam path to collect x-ray fluorescence, instead of taking direct images in the shadowgraphic mode as in CT. A schematic diagram is shown in FIG. 11. This figure shows how the assembly of the x-ray detector and source is moved around the imaging object after each scan. The distribution of the nanomaterial radiosensitizers in the body can be mapped out by computers after the scanning is complete. The x-ray fluorescence is emitted from the NEXT agents after absorption of scanning x-rays by the nanomaterials. The main benefit of this technique is that the detection sensitivity is enhanced.

Sensitive, large solid angle x-ray detectors (photon counting) can be used to detect characteristic emission from the elements (K lines from Au, Bi, Pt, or Gd) used in the sensitizers. Because a CAT scan can be used to deliver x-ray radiation, it is possible to map out the distribution of radiosensitizers in the body. In a CAT scan, a beam of conically shaped x-rays passes through a object of interest and is detected by an area x-ray detector. The orientation of the source-detector assembly is changed and scanned, and a series of planar images are obtained. A computer program is then used to reconstruct a three-dimensional image. Because the planar images are obtained according to the absorption of x-rays, radiosensitizers can be used to enhance the absorption. Even more sensitive is to detect x-ray fluorescence as a result of the absorption, which closely resembles that used in angiographs with radionuclides. However, radioactive elements are used in angiographs, thus posing health problems if the location of radionuclides cannot be precisely controlled. Using collimated external x-rays to activate nanomaterial radiosensitizers, a double-latched safety guard, affords both high sensitivity and low risk. A schematic diagram to describe this technique is given in FIG. 11.

Electromagnetic Radiation

Generally, any electromagnetic radiation may be used that causes release of Auger or other secondary electrons from the nanomaterials. Another common term for this type of radiation is ionizing radiation, implying the radiation can ionize the electrons of atoms. To cause release of Auger or other secondary electrons from nanomaterials in the body, the electromagnetic radiation must be able to penetrate the subject being treated to expose the nanomaterials associated with the targeted cells. Means of achieving this include but are not limited to using electromagnetic radiation of wavelengths not substantially absorbed by bodily tissues and fluids. Because of the prevalence of water in bodily tissues and fluids, electromagnetic radiation of wavelengths not substantially absorbed by water may be particularly useful. Examples of electromagnetic radiation that may be used with Gold nanoparticles is high energy x-ray radiation, including but not limited to x-ray radiation of energy greater than about 80.7 keV. The energy requirement for Gd is greater than 50.3 keV, and that for Bi is greater than 90.6 keV.

In the latest Computed Tomography (CT) technology, high energy x-ray sources and area detectors are used. The energy of these x-rays is between 10 to 400 keV. For example, a 200 kV rotating anode x-ray source is used in Feinfocus FXE 200.20 (FEINFOCUS GmbH, Germany). This energy is suitable for use with NEXT technology, as shown in FIG. 11. The absorption of x-rays by gold in this technique is still 10 times higher than that of water, making it useful for the practice of the present invention.

External ionizing radiation can be delivered to the target. Multiple collimated x-ray beams such as those used in Gamma knife technology or radiosurgery can be used. In Gamma knife technology or radiosurgery, several or more collimated x-ray beams can be focused to a single spot in the body to cause damage to the tissues or other organs in the region. The x-ray source can be rotating anodes or linear accelerators. The invention described here can be used with either rotating anode x-ray sources using a suitable target or table-top accelerators (Gibson et al., 2004).

Detection of Auger Electrons

A critical link in the chain of events after x-ray absorption by nanomaterial radiosensitizers is the generation of Auger electrons. It would therefore be useful to quantitatively measure the yield of Auger electrons. One method to increase the detection limit of Auger electrons is to use composite materials such as nanoparticle-nanowire complexes. The electrons so generated are expected to be injected into the conducting nanowires, and voltage signal changes will be detected.

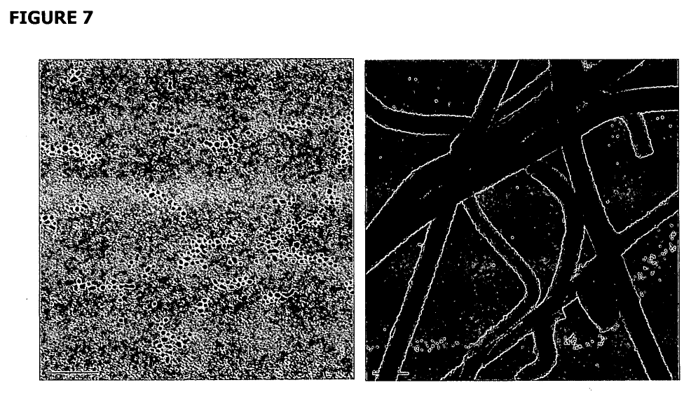

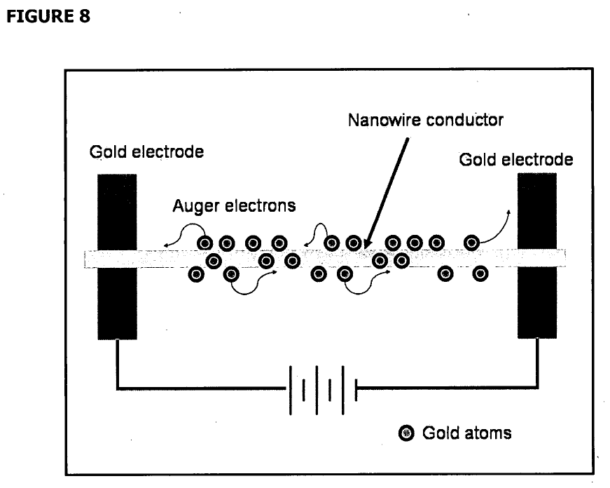

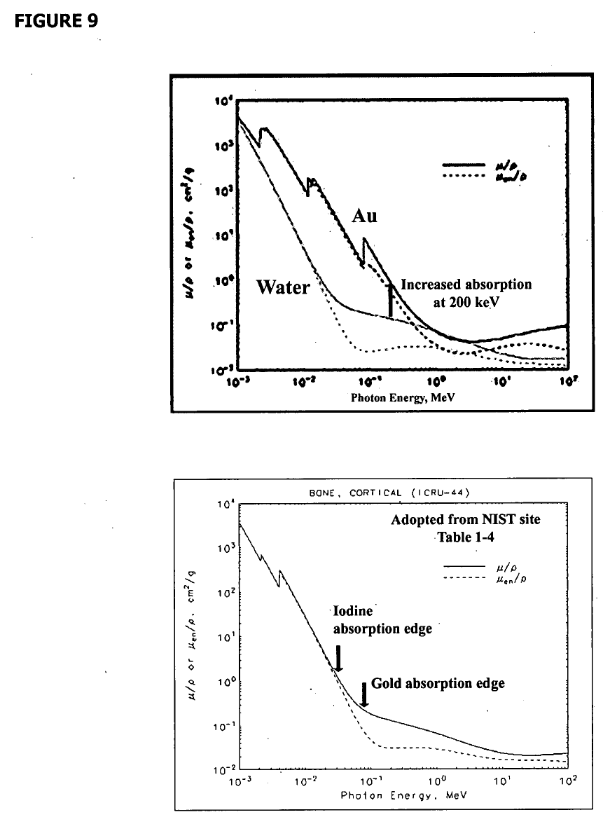

Another method is to coat gold nanoparticles onto silicon-based nanowires. FIG. 7 shows the results of attaching gold nanoparticles to silicon-based nanowires. The detection of Auger electrons can be realized if the nanowires are conductive, which can be CoSi₂ nanowires, or even single-walled carbon nanotubes. A device concept is shown in FIG. 8. In this design, Auger and other charges produced next to the conducting nanowires will inject the charges into the nanowire, which cause the electrical signal to change across the electrodes.

The invention will be better understood by reference to the following non-limiting example.

### EXAMPLE 1

### The Use of Gold Nanoparticles to Enhance X-Ray Absorption

Materials and Methods

Synthesis of TMAₙAuNP:

Dodecanethiol (C₁₂) functionalized gold nanoparticles (AuNP), NNN trimethyl(11-mercaptoundecyl) ammonium chloride ligands, and trimethylammonium (TMA) C₁₂ functionalized gold nanoparticles (TMAₙAuNP, n denotes the number of TMA ligands on a nanoparticle) were synthesized using the available procedures (Tien et al., 1997, Brust et al., 1994, McIntosh et al., 2001). Different reaction times (24-hour or 51-hour) were used in the ligand exchange reactions to make TMAₙAuNP, which yielded different n values. NMR and UV-VIS were used to verify the reaction intermediates and products.

Gel Electrophoresis

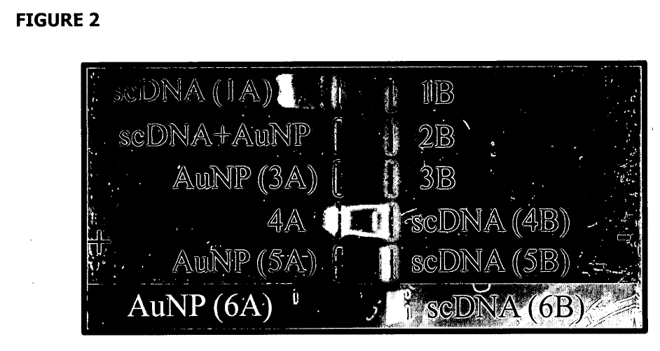

1.2% and 0.8% Agarose E-gels (Invitrogen) and Agarose gels prepared in the lab were used to detect supercoiled DNA, gold nanoparticles, and their complexes. The running conditions were between 50 or 60 V for 45 min (E-gels) or 3 hours (self-poured gels). Gels were inspected with a transilluminator (ChemiDoc XRS, Bio-Rad). In FIG. 2, two parallel columns of wells, 6 mm (w)×2 mm (thickness)×3 mm (depth) in dimension and labeled as 1A through 6A and 1B through 6B were prepared. The locations of scDNA were detected by adding ethidium bromide to the gels and viewed on the transilluminator.

Nanoparticle Inspections and Radiation Testing Conditions

A transmission electron microscope (TEM, Philips CM-12) was used to examine the nanoparticle samples. X-ray radiation tests were performed at the UC Davis Cancer Center (RS2000, Radsource, operated at 100 keV). The maximum dosage was determined by radiating free scDNA, normally at 0.5 Gy/min radiation flux. 0 to 16 minutes of radiation assays were used, with the 16-min radiation (8 Gy total dosage) being able to completely convert scDNA into relaxed scDNA.

Results

Trimethylammonium (TMA) C₁₂ functionalized gold nanoparticles (TMAₙAuNP, n denotes the number of TMA ligands on a nanoparticle) were synthesized. FIG. 1 shows a TEM image of the TMAₙAuNP (left panel) and the size distribution (right panel). The average size of 5 nm was slightly larger than that was made using the established procedure. Atomic force microscope (AFM) inspections showed a similar size distribution, which peaked at 5 nm. These nanoparticles were used in all measurements described below.

FIG. 2 shows results of gel electrophoresis experiments designed to probe the interactions and mobility of scDNA and TMAₙAuNP. TMAₙAuNP were added into wells 2A, 3A, 5A, and 6A, and scDNA samples were added into wells 1A, 2A, 4B, 5B, and 6B. Wells 1B, 2B, 3B, and 4A were left empty.

Since TMAₙAuNP (n≠0) are positively charged and scDNA are negatively charged in 1×TBE buffer, they moved in opposite directions in the gel, as shown in FIG. 2. TMAₙAuNP were visible as the stains spread to the right hand side of wells 2A, 3A, 5A, and 6A., indicating a distribution in the numbers of the TMA ligands on AuNP.

The migration of the scDNA in the wells of the right column in FIG. 2 was impeded by the presence of TMAₙAuNP travelling in the opposite direction in the same lanes. An example is given in lane 5, which shows that the scDNA was stopped well short of the normal distance travelled by the scDNA alone, as in lane 1 and 4. In lane 6, the distance between the two wells was increased, and the scDNA travelled further than that in lane 5. An extreme case is shown in lane 2, in which the scDNA did not move out of the well 2A when it was mixed with TMAₙAuNP at a high nanoparticle-to-DNA ratio (˜1000:1). In this case, the AuNP stain was still visible to the right of the well because of the large quantity used. The decreased mobility of the scDNA in the gel can be explained by the interactions of highly charged TMAₙAuNP with the scDNA.

Using the ratio of the distance traversed by the majority of TMAₙAuNP to that by the fastest moving TMAₙAuNP in lane 6, the upper limit of the number of TMA ligands on majority of the AuNP was estimated to be only 10% of the number of TMA ligands on the fastest moving AuNP. Assuming that there were 300 thiol ligands covering 900 surface Au atoms in a 5-nm nanoparticle, and with nearly 100 of those thiols being TMA ligands (the rest being dodecanethiol ligands), average AuNP had only ˜10-15 TMA ligands on them.

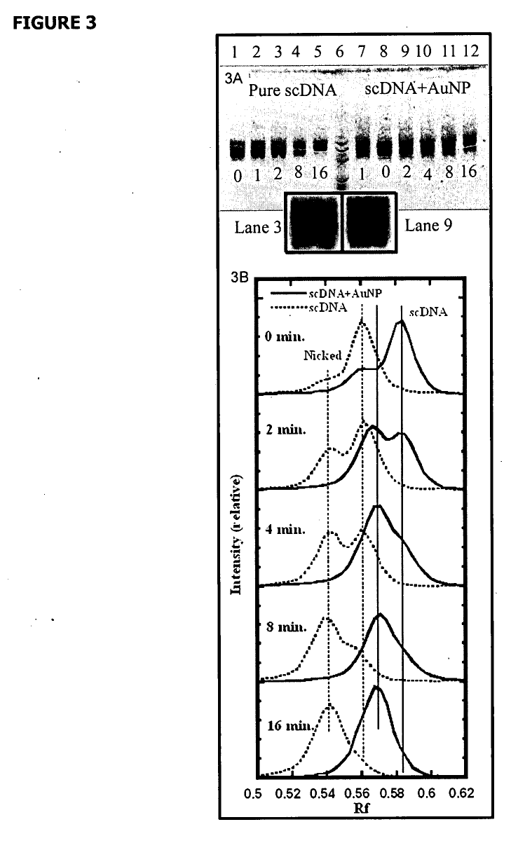

FIG. 3 shows the gel electrophoresis results of radiation tests. scDNA and scDNA-TMAₙAuNP complexes were pipetted into the lanes of the gels. Three pairs of 6-set samples were prepared. 200-ng scDNA was used for preparing each of the 36 samples. 1-μg 24-hour TMAₙAuNP was then added to each of the scDNA solutions to prepare the 18 scDNA-TMAₙAuNP samples. Each pair of samples were identically prepared and radiated. For each pair of the 6-set free scDNA and scDNA-TMAₙAuNP samples, they were injected into PCR tubes with the tabs removed and covered with 3-μm Mylar films, and were radiated for 0, 1, 2, 4, 8 and 16 minutes respectively. The experimental conditions such as preparation times, exact radiation times, and waiting times before and after the radiation varied slightly for these three pairs of samples. The samples were then loaded into the wells of the E-gels for quantitative analysis.

FIG. 3A shows the results of radiation tests, using samples of ˜100:1 TMAₙAuNP-to-scDNA ratio. The scDNA occupied the spots further down the lanes, trailed by the relaxed scDNA.

After radiation, the amounts of the relaxed scDNA for scDNA-TMAₙAuNP mixtures increased. For example, both samples in lanes 3 and 9 in the inserts of FIG. 3A had the same radiation time of 2 minutes: In lane 9 almost half the scDNA were in the relaxed or circular form, whereas less than 25% of the scDNA were in this form in lane 3.

A lineout plot of the results of another gel is shown in FIG. 3B. The samples were prepared similarly to those used in FIG. 3a. As shown, there was little additional relaxation (˜5%) caused by the presence of TMAₙAuNP (top panel in FIG. 3B). In contrast, the extent of the relaxation was almost the same for scDNA exposed for 4 min of radiation (dashed line) and for scDNA-TMAₙAuNP exposed for 2 min (solid line).

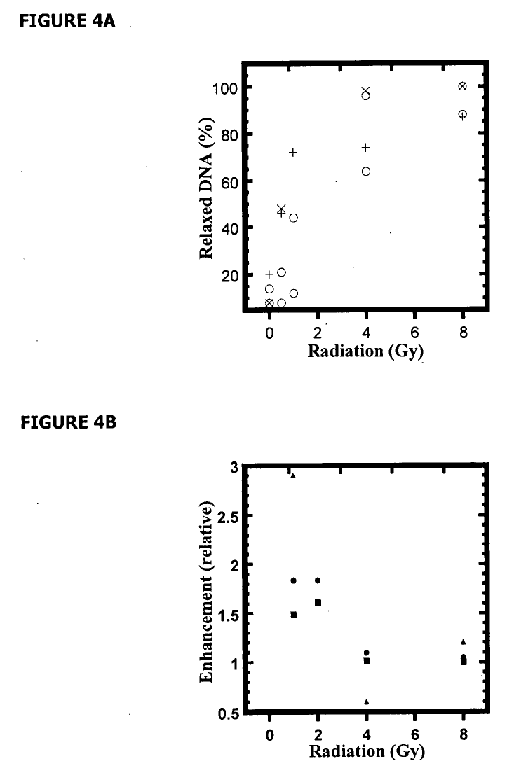

Three pairs of 6-set scDNA and 6-set scDNA-TMAₙAuNP samples were studied with radiation. Each pair of samples were identically prepared and radiated, whereas the experimental conditions for different pairs varied slightly. FIG. 4A shows the results of these radiation tests on the three pairs of free scDNA (empty symbols) and TMMuNP-bound scDNA (corresponding solid symbols) samples. Depending on the experimental conditions, the percentage of the relaxed scDNA varied, although there were always more relaxed scDNA for radiated scDNA-TMAₙAuNP samples within each pair.

The maximum enhancement was observed between 0.5 and 2 Gy of radiation for the scDNA used here. The relative enhancement ratio, which is the ratio of the percentage of the relaxed DNA in the AuNP-bound scDNA to that of the relaxed DNA in free scDNA in each of the pair samples, is plotted in FIG. 4B. The ratios were calculated after corrections of relaxation prior to radiation. The maximum enhancement was of the order of ˜200% (three times that of the free scDNA) and occurred between 0.5 and 2 Gy of radiation dosage. The average enhancement factor was ˜2.1 at 1 Gy. As high as 8 times enhancement at 0.5 Gy was observed (not shown).

It is necessary to explain why scDNA-TMAₙAuNP mixtures appeared at the same position as the free scDNA in the gel. Based on the results shown in FIG. 2, it is known that a majority of the TMAₙAuNP had only a few TMA ligands on them. It is then reasonable to assume that most of the TMAₙAuNP were separated from scDNA at the onset of the gel runs by the electrostatic forces exerted on the AuNP-scDNA complexes. For those with larger n, each TMAₙAuNP might form multiple electrostatic contacts with one or more scDNA, and migrated in the Agarose network at slower speeds. This is actually visible in FIG. 3a: the additional scDNA appeared as streaks trailing the main bands.

One can estimate the maximum theoretical enhancement factor by comparing the x-ray absorption cross-sections for the gold nanoparticles and water at 81 keV. Assuming that a scDNA occupies a 1.5-nm diameter cylinder, 500-nm long, and is surrounded by a 12-nm diameter cylinder of water, a distance over which Auger or other secondary electrons or hydroxyl radicals are effective, the x-ray absorption cross-section for this amount of water surrounding the scDNA is 0.18 (cm²/g, mass absorption coefficient at 82 keV)×1 g/cm³×(6×10⁻⁷)²×π×500×10⁻⁷=1.0×10⁻¹⁷ cm². The absorption cross-section for one hundred 5-nm gold nanoparticles (each contains ˜3300 Au atoms) decorated the scDNA is 7.9 (cm²/g)×196 amu×900 (number of surface atoms)×100 (number of gold nanoparticles)×1.6×10⁻²⁴ (g/amu)=2.2×10⁻¹⁶ cm² (Benfield, 1992)

Since only half of the Auger or other secondary electrons escaped from the AuNP face the scDNA, the effective absorption is half the value shown above. Therefore, 100 AuNP next to the scDNA are about 11 times as efficient as the 1.8 million water molecules around the scDNA.

In conclusion, these experiments showed a 100% enhancement of relaxation for nanoparticle-bound supercoiled DNA (scDNA) in aqueous solution after exposure to hard x-ray radiation.

### EXAMPLE 2

### Use of Gold Nanoparticles for Local Enhancement

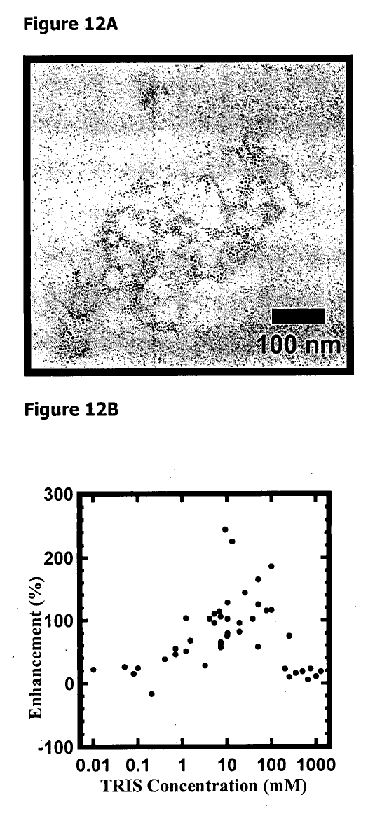

Gold nanoparticles conjugated to scDNA using an ethidium-based intercalating ligand were used. In this experiment, 3-nm gold nanoparticles covered with a mixture of ethidium thiol ligands (<10 per nanoparticle) and charge-neutral surfactants were prepared. The charge-neutral ligands were used to avoid any DNA aggregation. Such a small amount of ethidium in the samples (<150 nM) did not result in any detectable change to the scDNA. Tris buffer was added to control the diffusion distance of hydroxyl radicals in water (Hodgkins et al., 1996). The ratio of nanoparticles to scDNA was ˜10. FIG. 12A shows a transmission electron microscope (TEM) image of nanoparticle-conjugated scDNA in which all the nanoparticles are conjugated to scDNA. FIG. 12B shows the results of DNA damage as a function of buffer concentration, which directly controlled the diffusion distance of OH radicals. At low buffer concentrations the enhancement was nearly zero because radicals generated from background water diffused to scDNA to cause majority of the damage. The highest enhancement occurred when the electron migration distance was approximately the same as the radical diffusion distance, which was estimated to be 15-5 nm at 10-100 mM Tris buffer. When Tris concentration was too high, it even reduced the amount of radicals generated locally from nanoparticles near the target, thus reducing the enhancement. Because of the small amount of gold nanoparticles used here, remote damage by high energy photoelectrons emitted from these nanoparticles was negligible. This result also suggested that contribution from direct electron ionization/attachment by electrons released from these ten nanoparticles per scDNA was negligible.

### EXAMPLE 3

### Use of Gold Nanotubes for Remote Enhancement

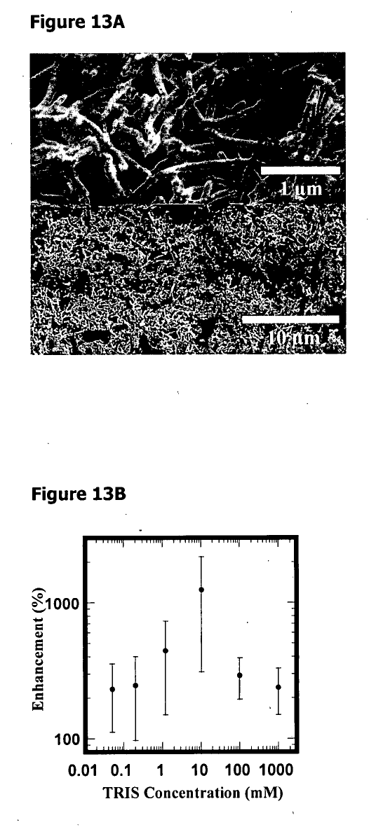

Remote enhancement may be demonstrated using gold nanotubules (Qu et al, 2006). In this demonstration, scDNA was mixed with a matrix of ˜20 mg ligand-free gold nanotubules in 20 μl of water, as shown in FIG. 13A. The ratio of the weight of these gold nanotubules to water in the scDNA samples was ˜1:1. Because the shell thickness of the gold layer was about 40 to 70 nm, only high energy photoelectrons could escape the nanostructures. The gaps between these nanotubules were of the order of several hundred nanometers and scDNA could easily move through the matrix. This creates a mismatch between the penetration distance (many microns) of high energy photoelectrons and the distance between the nanotubules, which leads to the re-absorption of those photoelectrons by gold nanotubules. A factor 2 reduction in enhancement was found with the gold nanotubule case because many low energy electrons cannot escape the nanotubules. The re-absorption further reduced the enhancement. After radiation, the aqueous scDNA was extracted with a pipet and detected with Agarose gel electrophoresis. FIG. 13B show that up to 2× enhancement of damage to scDNA was achieved at low Tris concentrations. As the Tris concentration increases, even though it may approximately affect equally the radicals generated from high energy photoelectrons from water and those from gold nanotubules, it does reduce the amount of radicals generated from Compton electrons generated in water because these Compton electrons have a maximum energy of 10 keV for the tungsten x-ray source we used. As a result, the maximum enhancement occurred at a lower Tris buffer concentration between 1-10 mM, smaller than the 10-100 mM Tris concentration used in the nanoparticle-induced local enhancement case (see FIGS. 12B and 13B).

### REFERENCES

The following references cited herein are hereby incorporated by reference in their entirety.
Agarwal, B. K. X-ray Spectroscopy, 2nd ed.; Springer-Verlag: New York, 1991; Vol. 15.
Aldrich et al, 42(2), 2410-2415, 1992
Alivisatos, A. P.; Johnsson, K. P.; Peng, X. G.; Wilson, T. E.; Loweth, C. J.; Bruchez, M. P.; Schultz, P. G. Organization of Nanocrystal Molecules Using DNA, Nature 1996, 382, 609-611.
L. Balogh, Bielinska, A., Eichman, J., Valluzzi, R., Lee, I., Baker, J., Lawrence, T. and Khan, M. Dendrimer nanocomposites in medicine. Chimica OGGI-Chemistry Today. 20. 35-40 (2002).
R. Barth, Coderre, J., Vicente, M. and Blue, T. Boron neutron capture therapy of cancer: Current status and future prospects. Clinical Cancer Research. 11. 3987-4002 (2005).
Benfield, R. E., Journal of the Chemical Society-Faraday Transactions, 1992, 88, 1107-1110
Boudaiffa, B.; Cloutier, P.; Hunting, D.; Huels, M. A.; Sanche, L. Low-energy electrons induced DNA strand breaks, M S-Medecine Sciences 2000, 16, 1281-1283.
Bramlett et al, Journal of Pharmacology and Experimental Therapeutics, 307, 291-296, 2003
M. Brust, M. Walker, D. Bethell, D. J. Schiffrin and R. Whyman, Journal of the Chemical Society-Chemical Communications, 1994, 801-802
P. E. Bryant. Enzymatic restriction of mammalian cell DNA: evidence for the double strand breaks as potentially lethal lesions. Int J Radiat Biol 48. 55-60 (1984).
L. Brown and Hutchison, J. Controlled growth of gold nanoparticles during ligand exchange. JACS. 121. 882-883 (1999).
Chariton, D. E.; Booz, J. A. A Monte Carlo Treatment of the Decay of 125I, Radiation Research 1981, 87, 10-23.
D. E. Charlton and Humm, J. L. A method of calculating initial DNA strand breakage following the decay of incorporated 125I. Int. J. Radiat Biol. 53. 353-365 (1988).
Chen, P.; Cameron, R.; Wang, J.; Vallis, K. A.; Reilly, R. M. Antitumor effects and normal tissue toxicity of In-111-labeled epidermal growth factor administered to athymic mice bearing epidermal growth factor receptor-positive human breast cancer xenografts, Journal of Nuclear Medicine 2003, 44, 1469-1478.
J. Clark, Fronczek, F. and Vicente, M. Novel carboranylporphyrins for application in boron neutron capture therapy (BNCT) of tumors. Tetrahedron Letters. 46. 2365-2368 (2005).
D. Cliffel, Zamborini, F., Gross, S. and Murray, R. Mercaptoammonium-monolayer-protected, water-soluble gold, silver, and palladium clusters. Langmuir. 16. 9699-9702 (2000).
L. Cognet, Tardin, C., Boyer, D., Choquet, D., Tamarat, P. and Lounis, B. Single metallic nanoparticle imaging for protein detection in cells. PNAS. 100. 11350-11355 (2003).
Fairchild, R. G.; Bond, V. P. Photon activation therapy, Strahlentherapie 1984, 160, 758-63.
E. Foley, Carter, J., Shan, F. and Guo, T. Enhanced relaxation of nanoparticle-bound supercoiled DNA in X-ray radiation. CHEMICAL COMMUNICATIONS. 3192-3194 (2005).
Foos et al, 14, 2401-2408, 2002
B. L. Franc, S. J., M., Z., S., P., W. and C. H., C. Breaching biological barriers: protein translocation domains as tools for molecular imaging and therapy. Mol. Imaging. 2. 313-323 (2003).
Ghetie, M., Picker, L., Richardson, J., Tucker, K., Uhr, J., and Vitetta, E. Anti-CD19. Inhibits the Growth of Human B-cell Tumor Lines In vitro and of Daudi cells in SCID Mice by Inducing Cell-Cycle Arrest. Blood. 83. 1329-1336 (1994).
Gibson et al, Phys. Plasmas, 11, 2857-2864, 2004.
Hall, E. J. Radiobiology for the Radiologist, 1st ed.; Harper & Row, Publishers: New York, 1973.
Hofer, K. G. Biophysical aspects of Auger processes, Acta Oncologica 2000, 39, 651-657.
K. G. Hofer, Prensky, W. and Hughes, W. L. Death and metastastic distribution of tumor cells in mice monitored with 125I-iododeoxyuridine. J. Nat. Cancer Inst. 43. 763-773 (1969).
M. A. Huels, Boudaiffa, B., Cloutier, P., Hunting, D. and Sanche, L. Single, double, and multiple double strand breaks induced in DNA by 3-100 eV electrons. Journal of the American Chemical Society. 125. 4467-4477 (2003).
Karnas, S. J.; Moiseenko, V. V.; Yu, E.; Truong, P.; Battista, J. J. Monte Carlo simulations and measurement of DNA damage from x-ray-triggered Auger cascades in iododeoxyuridine (IUdR), Radiation and Environmental Biophysics 2001, 40, 199-206.
Kassis, A. I. Cancer therapy with Auger electrons: Are we almost there?, Journal of Nuclear Medicine 2003, 44, 1479-1481.
Z. Kaul, Yaguchi, T., Kaul, S., Hirano, T., Wadhwa, R. and Taira, K. Mortalin imaging in normal and cancer cells with quantum dot immuno-conjugates. Cell Research. 13. 503-507 (2003).
Kinsella, T.; Collins, J.; Rowland, J. Pharmacology and phase I/II study of continuous intravenous infusions of iododeoxyuridine and hyperfractionated radiotherapy in patients with glioblastoma multiforme, J. Clin. Oncol. 1988, 6, 871-879.
Kobayashi, K.; Usami, N.; Sasaki, I.; Frohlich, H.; Le Sech, C. Study of Auger effect in DNA when bound to molecules containing platinum.A possible application to hadrontherapy, Nuclear Instruments & Methods in Physics Research Section B-Beam Interactions With Materials and Atoms 2003, 199, 348-355.
J. Kukowska-Latallo, Candido, K., Cao, Z., Nigavekar, S., Majoros, I., Thomas, T., Balogh, L., Khan, M. and Baker, J. Nanoparticle targeting of anticancer drug improves therapeutic response in animal model of human epithelial cancer. Cancer Research. 65. 5317-5324 (2005).
Laster, B. H.; Thomlinson, W. C.; Fairchild, R. G. Photon Activation of Iododeoxyuridine: Biological Efficacy of Auger Electrons, Radiation Research 1993, 133, 219-224.
M. Lindgren, Gallet, X., Soomets, U., Hallbrink, M., Brakenhielm, E., Pooga, M., Brasseur, R. and Langel, U. Translocation properties of novel cell penetrating transportan and penetratin analogues. Bioconjugate Chemistry. 11. 619-626 (2000).
C. Loo, Lowery, A., Halas, N., West, J. and Drezek, R. Immunotargeted nanoshells for integrated cancer imaging and therapy. Nano Letters. 5. 709-711 (2005).
D. Matthews, Appelbaum, F., Eary, J., Mitchell, D., Press, O. and Bernstein, I. Phase I study of I-131-anti-CD45 antibody plus cyclophosphamide and total body irradiation for advanced acute leukemia and myelodysplastic syndrome. Blood. 90. 1854-1854 (1997).
M. McDevitt, Barendswaard, E., Ma, D., Lai, L., Curcio, M., Sgouros, G., Ballangrud, A., Yang, W., Finn, R., et al. An alpha-particle emitting antibody ([Bi-213]J591) for radioimmunotherapy of prostate cancer. Cancer Research. 60. 6095-6100 (2000).
McIntosh, C. M., E. A. Esposito, A. K. Boal, J. M. Simard, C. T. Martin and V. M. Rotello, Journal of the American Chemical Society, 2001, 123, 7626-7629
Moss, W. T.; Cox, J. D. Radiation Oncology. Rationale, Technique, Results, 7th ed.;
The C.V. Mosby Company: St. Louis, 2003.
E. Neuwelt, Varallyay, P., Bago, A., Muldoon, L., Nesbit, G. and Nixon, R. Imaging of iron oxide nanoparticles by MR and light microscopy in patients with malignant brain tumours. Neuropathology and Applied Neurobiology. 30. 456-471 (2004).
S. Nigavekar, Sung, L., Llanes, M., El-Jawahri, A., Lawrence, T., Becker, C., Balogh, L. and Khan, M. H-3 dendrimer nanoparticle organ/tumor distribution. Pharmaceutical Research. 21. 476-483 (2004).
B. Nikoobakht and El-Sayed, M. Preparation and growth mechanism of gold nanorods (NRs) using seed-mediated growth method. Chemistry of Materials. 15. 1957-1962 (2003).
Nutt et al, Environmental Sci. Technology, 39, 1346-1353, 2005
T. Pellegrino, Manna, L., Kudera, S., Liedl, T., Koktysh, D., Rogach, A., Keller, S., Radler, J., Natile, G., et al. Hydrophobic nanocrystals coated with an amphiphilic polymer shell: A general route to water soluble nanocrystals. NANO LETTERS. 4. 703-707 (2004).
Pignol, J. P.; Rakovitch, E.; Beachey, D.; Le Sech, C. Clinical significance of atomic inner shell ionization (ISI) and Auger cascade for radiosensitization using IUdR, BUdR, platinum salts, or gadolinium porphyrin compounds, International Journal of Radiation Oncology, Biology, Physics 2003, 55, 1082-1091.
M. Pooga, Kut, C., Kihimark, M., Halibrink, M., Fernaeus, S., Raid, R., Land, T., Hallberg, E., Bartfai, T., et al. Cellular translocation of proteins by transportan. FASEB J. 15.-(2001).
M. Rojas, Donahue, J., Tan, Z. and Lin, Y. Genetic engineering of proteins with cell membrane permeability. Nature Biotechnology. 16. 370-375 (1998).
Sandstrom, P.; Boncheva, M.; Akerman, B. Nonspecific and thiol-specific binding of DNA to gold nanoparticles, Langmuir 2003, 19, 7537-7543.
Schmid G., Chem Rev, 92, 1709, 1992
A. Schroedter and Weller, H. Ligand design and bioconjugation of colloidal gold nanoparticles. Angewandte Chemie-International Edition. 41. 3218-+ (2002).
Seino et al, Journal of Ceramic Processing Research, 5(2), 136-139, 2004
Shenhar, R.; Rotello, V. M. Nanoparticles: Scaffolds and building blocks [Review], Accounts of Chemical Research 2003, 36, 549-561.
A. Smith and Nie, S. Chemical analysis and cellular imaging with quantum dots. Analyst 129. 672-677 (2004).
Sovico et al, Bioconjugate Chem., 16(5), 1181-1188, 2005.
Stella et al, J. Pharm. Sci., 89(11), 1452-1464, 2000.
Tien, J., A. Terfort and G. M. Whitesides, Langmuir, 1997, 13, 5349-5355.
Y. Tseng, Liu, J. and Hong, R. Translocation of liposomes into cancer cells by cell-penetrating peptides penetratin and TAT: A kinetic and efficacy study. Molecular Pharmacology. 62. 864-872 (2002).
von Sonntag, C. The Chemical Basis for Radiation Biology, Taylor and Francis: London, 1987.
Walicka, M. A.; Ding, Y.; Adelstein, S. J.; Kassis, A. I. Toxicity of DNA-incorporated iodine-125: Quantifying the direct and indirect effects, Radiation Research 2000, 154, 326-330.
Walter, P. and Blobel, G. Translocation of Proteins Across the Endoplasmic Reticulum 2: Signal Recognition Protein (SRP) Mediates the Selective Binding to Microsomal Membranes of In vitro Assembled Polysomes Synthesizing Secretory Protein. Journal of Cell Biology. 91. 551-556 (1981).
Wang et al, Anal. Chem., 74, 4320-4327, 2002.
Wilcoxon et al, J. Phys. Chem. B, 103, 9809-9812, 1999.
P. Winter, Morawski, A., Caruthers, S., Harris, T., Fuhrhop, R., Zhang, H., Allen, J., Lacy, E., Williams, T., et al. Paramagnetic a(v)b(3)-integrin-targeted fumagillin nanoparticles for combined molecular imaging and antiangiogenic therapy in atherosclerosis. Circulation. 110. 306-306 (2004).
Zanchet, D.; Micheel, C. M.; Parak, W. J.; Gerion, D.; Williams, S. C.; Alivisatos, A. P. Electrophoretic and structural studies of DNA-directed Au nanoparticle groupings, Journal of Physical Chemistry B 2002, 106, 11758-11763.
Zhao et al, Apoptosis, 8, 631-637, 2004
A. Ziegler, Nervi, P., Durrenberger, M. and Seelig, J. The cationic cell-penetrating peptide Cpp(TAT) derived from the HIV-1 protein TAT is rapidly transported into living fibroblasts: Optical, biophysical, and metabolic evidence. Biochemistry. 44. 138-148 (2005).
P. Hodgkins, Fairman, M. and ONeill, P. Rejoining of gamma-radiation-induced single-strand breaks in plasmid DNA by human cell extracts: Dependence on the concentration of the hydroxyl radical scavenger, tris. Radiation Research 145. 24-30 (1996).
Y. Qu, Carter, J., Sutherland, A. and Guo, T. “Surface modification of gold nanotubules via microwave radiation, sonication and chemical etching,” Chemical Physics Letters. 432. 195-199 (2006).
Y. Qu, Porter, R., Shan, F., Carter, J. and Guo, T. “Synthesis of tubular gold and silver nanoshells using silica nanowire core templates,” Langmuir. 22. 6367-6374 (2006).

We claim:

1. A method of inducing damage to a molecule, comprising the steps of:
delivering a nanomaterial comprising at least one targeting moiety capable of binding to the molecule to a location that is 5-10 nm or less in distance from the molecule; and
exposing the nanomaterial to electromagnetic radiation under conditions wherein the nanomaterial releases electrons that directly or indirectly induce damage to the molecule.

2. The method of claim 1, wherein the targeting moiety is no greater than 10 nm in length.

3. The method of claim 1, wherein the nanomaterial also induces damage to one or more additional molecules within approximately 5-10 nm of the nanomaterial.

4. The method of claim 1, wherein the nanomaterial is selected from the group consisting of a nanoparticle, a nanorod, a nanoshell, a nanowire, a nanotube, a nanoparticle-nanorod complex, a nanoparticle-nanowire complex, a patterned nanoparticle complex connected by ligands, and a silicon-based nanowire (SiNW).

5. The method of claim 1, wherein the nanomaterial is 1 to 1000 nm in size.

6. The method of claim 1, wherein the target molecule is DNA or protein.

7. The method of claim 1, wherein the targeting moiety is selected from the group consisting of: DNA, antibody, cell penetrating peptide, translocation protein, ethidium ligand, thiol ligand, phosphane ligand, signal peptide sequence, super-antibody, chemotherapeutic agent, low density lipoprotein, capillary-binding molecule, and polymer.

8. The method of claim 1, wherein the nanomaterial comprises a surface functionalization ligand, wherein the surface functionalization ligand provides a function selected from the group consisting of increasing water solubility, increasing biocompatibility, increasing fat solubility, and increasing stability in an acidic environment.

9. The method of claim 1, wherein said electromagnetic radiation is X-ray radiation; wherein said X-ray radiation has energy greater than about 50.3 keV, or greater than about 80.7 keV.

10. The method of claim 1, wherein the nanomaterial comprises a heavy metal selected from the group consisting of gold, lanthanum (La), cerium (Ce), praseodymium (Pr), neodymium (Nd), promethium (Pm), samarium (Sm), europium (Eu), gadolinium(Gd), terbium (Tb), dysprosium (Dy), holmium (Ho), erbium (Er), thulium (Tm), ytterbium (Yb), lutetium (Lu), iodine, tungsten, rhenium, osmium, iridium, platinum, and bismuth.

11. The method of claim 1, wherein the molecule is in or on a cancer cell, a bacterium, or a virus.

12. The method of claim 11, wherein the molecule is in or on a cancer cell in an individual and the mass of the nanomaterial delivered is 0.01% to 0.0001% of the mass of the cancer cell in the individual.

13. The method of claim 1 wherein the nanomaterial is a gold nanoparticle, the electromagnetic radiation is X-ray radiation of energy greater than about 80.7 keV, and the molecule is DNA.

14. The method of claim 1 wherein the nanomaterial further includes a UV to IR chromophore or a UV to IR fluorophore.

15. A method of detecting a molecule, comprising:
delivering a nanomaterial comprising at least one targeting moiety capable of binding to the molecule to a location that is 10 nm or less in distance from the molecule;
exposing the nanomaterial to electromagnetic radiation under conditions wherein the nanomaterial emits radiation;
detecting the radiation with a detector to detect the molecule.

16. The method of claim 15 wherein the nanomaterial is selected from the group consisting of a nanoparticle, a nanorod, a nanoshell, a nanowire, a nanotube, a nanoparticle-nanorod complex, a nanoparticle-nanowire complex, a patterned nanoparticle complex connected by ligands, and a silicon-based nanowire (SiNW).

17. The method of claim 15 wherein the targeting moiety is selected from the group consisting of: DNA, antibody, cell penetrating peptide, translocation protein, ethidium ligand, thiol ligand, phosphane ligand, signal peptide sequence, super-antibody, chemotherapeutic agent, low density lipoprotein, capillary-binding molecule, and polymer.

18. The method of claim 15 wherein the nanomaterial comprises a surface functionalization ligand, wherein the surface functionalization ligand provides a function selected from the group consisting of increasing water solubility, increasing biocompatibility, increasing fat solubility, and increasing stability in an acidic environment.

19. The method of claim 15 wherein the nanomaterial comprises a heavy metal selected from the group consisting of gold, lanthanum (La), cerium (Ce), praseodymium (Pr), neodymium (Nd), promethium (Pm), samarium (Sm), europium (Eu), gadolinium(Gd), terbium (Tb), dysprosium (Dy), holmium (Ho), erbium (Er), thulium (Tm), ytterbium (Yb), lutetium (Lu), iodine, tungsten, rhenium, osmium, iridium, platinum, and bismuth.

20. The method of claim 15 wherein the nanomaterial is a gold nanoparticle and the electromagnetic radiation is X-ray radiation having energy greater than about 80.7 keV.
